(19) United States
(12) Patent Application Publication
(10) Pub. No.: US 2002/0039587 A1
(43) Pub. Date: Apr. 4, 2002

(54) Compositions and methods for treating hand and wrist discomfort

(76) Inventor: Yuanjin Tao, Fremont, CA (US)

Correspondence Address: MORRISON & FOERSTER LLP, 755 PAGE MILL RD, PALO ALTO, CA, 94304-1018, US

(21) Appl. No.: 09/873,175

(22) Filed: Jun. 1, 2001

Related U.S. Application Data
(60) Provisional application No. 60/208,991, filed on Jun. 1, 2000.

(51) Int. Cl. A61K 35/84; A61K 35/78

(52) U.S. Cl. 424/195.15; 424/725; 424/739; 424/764

(57) ABSTRACT

Compositions and methods for treating hand or wrist discomfort are provided. The compositions comprise singly or in combination herbals, natural nutritional supplements, minerals and vitamins. Methods of making these compositions are also provided.

### CROSS-REFERENCE TO RELATED APPLICATIONS

This application claims the benefit of U.S. Provisional Application Serial No. 60/208,991, filed Jun. 1, 2000, hereby incorporated in its entirety by reference.

### TECHNICAL FIELD

This invention relates to compositions and methods for treating hand and wrist disorders. More specifically, it relates to nutraceutical compositions for treating hand or wrist discomfort symptoms, and methods for treating these symptoms with said compositions.

### BACKGROUND ART

In an era of pervasive computer usage, the incidence of hand and wrist syndromes, including strain injuries such as carpal tunnel syndrome, arising from the repetitive motion nature of the use of computer keyboards is expected to increase. Indeed, carpal tunnel syndrome has reached epidemic proportions and has even been called the technological disease of the times. Szabo, R. M., Clin. Orthop. (1998), 351:78-89. Other repetitive strain injuries include syndromes such as tendinits, bursitis, tenosynovitis, DeQuervain, thoracic outlet, trigger finger/thumb and myofascial pain. These syndromes can cause great pain and permanent disability. Sternbach, G., J. Emerg. Med. (1999), 17(3):519-23; De Smet, L. et al., Acta. Orthop. Belg. (1995), 61(3):177-82; Abrams, R. A., and Gellman, H., Hand Clin. (1996), 12(4):731-43; Melhom, J. M., Kans. Med. (1995), 95(9):189-92; and Kuschner, S. H., et al., Orthopedics (1992), 15(11):1297-302.

A nutraceutical composition for relieving hand strain is known. (Capolla CHS Reliever, Amino Food Technologies, San Jose, Calif.). The ingredients in this formula include pyridoxine, thiamine, riboflavin, niacin, pantothenic acid, ascorbic acid, beta-carotene, natural vitamin E, zinc, selenium, astragalus extract, dong quai extract, devil's claw extract, ginger extract, cinnamomium extract, quercetin, bio-flavonoids and magnesium stearate.

A composition for treating degenerative musculoskeletal diseases such as rheumatoid arthritis and osteoarthritis is known. U.S. Pat. No. 5,494,668. Its ingredients include ashwagandha, sallai guggul, turmeric and ginger.

A composition for arresting the inflammatory response and facilitating repair and maintenance of damaged tissues in joints is known. U.S. Pat. No. 5,916,565. Its ingredients include a palatability agent, cayenne, ginger, tumeric, yucca, devil's claw, nettle leaf, black cohosh, alfalfa, celery seeds, D-glucosamin HCl and mucopolysaccharides.

A composition for the treatment of carpal tunnel syndrome is disclosed in U.S. Pat. No. 5,118,505. Its ingredients include the active substances of ginkgo bilobae extract and at least one substance of the group consisting of folic acid and its derivatives, alpha-lipoic acid and its derivatives, and all the vitamin B groups and their derivatives.

In view of the seriousness and prevalence of hand and wrist discomfort syndromes, and the corresponding need to alleviate said symptoms without significant side effects, it would be advantageous to have compositions and methods that are efficacious and safe to address this need. The invention described and claimed in this specification presents such compositions and methods.

All references cited herein, including patent applications and publications, are incorporated by reference in their entirety.

### DISCLOSURE OF THE INVENTION

Compositions and methods for treating hand or wrist discomfort are provided. The compositions comprise substances in quantities that are effective in relieving hand or wrist discomfort. Methods of treating hand or wrist discomfort comprising administration of the claimed compositions are also provided. Methods of making compositions of the invention are also provided.

Accordingly, in one aspect, the invention provides a composition for treating hand or wrist discomfort comprising 1 substance selected from the group consisting of a member of the botanical group Paeonia, a member of the botanical group Ligusticum, a member of the botanical group Ledebouriella, a member of the botanical group Cinnamomum, a member of the botanical group Carthamus and a member of the botanical group Corydalis.

In another aspect, the invention provides a composition for treating hand or wrist discomfort comprising 2 substances selected from the group consisting of a member of the botanical group Paeonia, a member of the botanical group Ligusticum, a member of the botanical group Ledebouriella, a member of the botanical group Cinnamomum, a member of the botanical group Carthamus and a member of the botanical group Corydalis.

In another aspect, the invention provides a composition for treating hand or wrist discomfort comprising 3 substances selected from the group consisting of a member of the botanical group Paeonia, a member of the botanical group Ligusticum, a member of the botanical group Ledebouriella, a member of the botanical group Cinnamomum, a member of the botanical group Carthamus and a member of the botanical group Corydalis.

In another aspect, the invention provides a composition for treating hand or wrist discomfort comprising 4 substances selected from the group consisting of a member of the botanical group Paeonia, a member of the botanical group Ligusticum, a member of the botanical group Ledebouriella, a member of the botanical group Cinnamomum, a member of the botanical group Carthamus and a member of the botanical group Corydalis.

In another aspect, the invention provides a composition for treating hand or wrist discomfort comprising 5 substances selected from the group consisting of a member of the botanical group Paeonia, a member of the botanical group Ligusticum, a member of the botanical group Ledebouriella, a member of the botanical group Cinnamomum, a member of the botanical group Carthamus and a member of the botanical group Corydalis.

In another aspect, the invention provides a composition for treating hand or wrist discomfort comprising a member of the botanical group Paeonia, a member of the botanical group Ligusticum, a member of the botanical group Ledebouriella, a member of the botanical group Cinnamomum, a member of the botanical group Carthamus and a member of the botanical group Corydalis.

In one embodiment, the invention provides a composition selected from the group consisting of the compositions of the aspects of the invention described in the preceding paragraphs, further comprising a substance selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium. Thus, in one embodiment, a composition may comprise 1, 2, or 3 substances selected from the group consisting of a member of the botanical group Ligusticum, a member of the botanical group Cinnamomum and a member of the botanical group Corydalis, further comprising a substance selected from the group consisting of a member of the botanical group Angelica, a member of the botanical group Astragalus and a member of the botanical group Morus.

In one embodiment, the invention provides a composition selected from the group consisting of the compositions of the aspects of the invention described in the preceding paragraphs, further comprising 2 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium. Thus, in one embodiment, a composition may comprise 1, 2, or 3 substances selected from the group consisting of a member of the botanical group Ligusticum, a member of the botanical group Cinnamomum and a member of the botanical group Corydalis, further comprising 2 substances selected from the group consisting of a member of the botanical group Angelica, a member of the botanical group Astragalus and a member of the botanical group Morus.

In one embodiment, the invention provides a composition selected from the group consisting of the compositions of the aspects of the invention described in the preceding paragraphs, further comprising 3 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium. Thus, in one embodiment, a composition may comprise 1, 2, or 3 substances selected from the group consisting of a member of the botanical group Ligusticum, a member of the botanical group Cinnamomum and a member of the botanical group Corydalis, further comprising 3 substances selected from the group consisting of a member of the botanical group Angelica, a member of the botanical group Astragalus and a member of the botanical group Morus.

In one embodiment, the invention provides a composition selected from the group consisting of the compositions of the aspects of the invention described in the preceding paragraphs, further comprising 4 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

In one embodiment, the invention provides a composition selected from the group consisting of the compositions of the aspects of the invention described in the preceding paragraphs, further comprising 5 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

In one embodiment, the invention provides a composition selected from the group consisting of the compositions of the aspects of the invention described in the preceding paragraphs, further comprising 6 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

In one embodiment, the invention provides a composition selected from the group consisting of the compositions of the aspects of the invention described in the preceding paragraphs, further comprising 7 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

In one embodiment, the invention provides a composition selected from the group consisting of the compositions of the aspects of the invention described in the preceding paragraphs, further comprising 8 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

In one embodiment, the invention provides a composition selected from the group consisting of the compositions of the aspects of the invention described in the preceding paragraphs, further comprising 9 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

In one embodiment, the invention provides a composition selected from the group consisting of the compositions of the aspects of the invention described in the preceding paragraphs, further comprising 10 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

In one embodiment, the invention provides a composition selected from the group consisting of the compositions of the aspects of the invention described in the preceding paragraphs, further comprising 11 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

In one embodiment, the invention provides a composition selected from the group consisting of the compositions of the aspects of the invention described in the preceding paragraphs, further comprising 12 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

In one embodiment, the invention provides a composition selected from the group consisting of the compositions of the aspects of the invention described in the preceding paragraphs, further comprising 13 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

In one embodiment, the invention provides a composition selected from the group consisting of the compositions of the aspects of the invention described in the preceding paragraphs, further comprising 14 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

In one embodiment, the invention provides a composition selected from the group consisting of the compositions of the aspects of the invention described in the preceding paragraphs, further comprising 15 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In one embodiment, the invention provides a composition selected from the group consisting of the compositions of the aspects of the invention described in the preceding paragraphs, further comprising 16 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

In one embodiment, the invention provides a composition selected from the group consisting of the compositions of the aspects of the invention described in the preceding paragraphs, further comprising 17 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

In one embodiment, the invention provides a composition selected from the group consisting of the compositions of the aspects of the invention described in the preceding paragraphs, further comprising 18 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

In one embodiment, the invention provides a composition selected from the group consisting of the compositions of the aspects of the invention described in the preceding paragraphs, further comprising 19 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

In one embodiment, the invention provides a composition selected from the group consisting of the compositions of the aspects of the invention described in the preceding paragraphs, further comprising 20 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

In one embodiment, the invention provides a composition selected from the group consisting of the compositions of the aspects of the invention described in the preceding paragraphs, further comprising a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

In various embodiments of the compositions of the invention described in the preceding and following paragraphs, the member of the botanical group Paeonia may be baishaoyao, the member of the botanical group Ligusticum may be chuanxiong, the member of the botanical group Ledebouriella may be fangfeng, the member of the botanical group Cinnamomum may be guizi, the member of the botanical group Carthamus may be honghua, and/or the member of the botanical group Corydalis may be yanhusuo. In some embodiments, the member of the botanical group Ligusticum may be chuanxiong, the member of the botanical group Ledebouriella may be fangfeng, the member of the botanical group Cinnamomum may be guizi, the member of the botanical group Carthamus may be honghua, the member of the botanical group Corydalis may be yanhusuo, the member of the botanical group Angelica may be dangquiwei, the member of the botanical group Glycyrrhiza may be gancao, the member of the botanical group Gingiberis may be ginger, the member of the botanical group Astragalus may be huangqi, the member of the botanical group Morus may be sangzhi, the member of the botanical group Epimedium may be yinyangho, the member of the botanical group Clematis may be weilingxian, and/or the member of the botanical group Zizyphus may be dazao.

Accordingly, in one aspect, the invention provides a composition for treating hand or wrist discomfort comprising a substance selected from the group consisting of baishaoyao, chuanxiong, fangfeng, guizi, honghua and yanhusuo.

In another aspect, the invention provides a composition for treating hand or wrist discomfort comprising 2 substances selected from the group consisting of baishaoyao, chuanxiong, fangfeng, guizi, honghua and yanhusuo.

In another aspect, the invention provides a composition for treating hand or wrist discomfort comprising 3 substances selected from the group consisting of baishaoyao, chuanxiong, fangfeng, guizi, honghua and yanhusuo.

In another aspect, the invention provides a composition for treating hand or wrist discomfort comprising 4 substances selected from the group consisting of baishaoyao, chuanxiong, fangfeng, guizi, honghua and yanhusuo.

In another aspect, the invention provides a composition for treating hand or wrist discomfort comprising 5 substances selected from the group consisting of baishaoyao, chuanxiong, fangfeng, guizi, honghua and yanhusuo.

In yet another aspect, the invention provides a composition for treating hand or wrist discomfort comprising baishaoyao, chuanxiong, fangfeng, guizi, honghua and yanhusuo.

In one embodiment, the invention provides a composition selected from the group consisting of the compositions of the aspects of the invention described in the preceding paragraphs, further comprising a substance selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 2 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 3 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 4 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 5 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 6 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 7 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 8 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 9 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 10 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 11 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 12 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 13 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 14 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 15 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 16 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 17 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 18 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 19 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising 20 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In another embodiment, the invention provides a composition selected from the group consisting of the compositions of the aspects of the invention described in the preceding paragraphs, further comprising dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

In one aspect, the invention provides a method of treating hand or wrist discomfort comprising administering to the individual an effective amount of a composition selected from the group consisting of the compositions of the aspects and embodiments of the invention described in the preceding and following paragraphs, whereby hand or wrist discomfort is treated.

In another aspect, the invention provides a method of making a composition for treating hand or wrist discomfort comprising combining at least two (preferably 2, 3, 4, 5, or 6) substances (preferably in an effective amount) selected from the group consisting of a member of the botanical group Paeonia, a member of the botanical group Ligusticum, a member of the botanical group Ledebouriella, a member of the botanical group Cinnamomum, a member of the botanical group Carthamus and a member of the botanical group Corydalis. In some embodiments of these methods, said at least two (preferably 2, 3, 4, 5, 6, 7, 8, 9, 10, 11, 12, 13, 14, 15, 16, 17, 18, 19, 20, or 21) substances (preferably in an effective amount) are combined with at least one substance selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium. In some embodiments, said combining is by mixing (such as by stirring, agitation or vibration). In some embodiments, the substances are packaged in the form of capsules, preferably in size “0”, “00”, “000”, “1”, “2”, “3” or “4.” In yet other embodiments, the substances are combined in powder form, preferably to at least 30%, 60%, or 90% mixture consistency, or to homogeneity.

### Modes for Carrying out the Invention

The present invention discloses compositions comprising single or multiple substances that are effective and safe for treating symptoms of hand or wrist discomfort. The invention further provides methods of treating hand or wrist discomfort using the disclosed compositions. Methods of making compositions of the invention are also provided.

### Definitions

The term “treating,” “treatment,” and variations thereof, as used in this specification, refers to an approach for obtaining beneficial or desired physiological results, which may be established clinically. For purposes of this invention, beneficial or desired clinical results include, but are not limited to, alleviation of symptoms, diminishment of extent of disease, stabilized (i.e., not worsening) condition, delay or slowing of progression or worsening of condition/symptoms, amelioration or palliation of the condition or symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering compositions of the present invention.

A “treatment effect” or “therapeutic effect” is manifested if there is a change in the condition being treated, as measured by the criteria constituting the definition of the terms “treating” and “treatment.” There is a “change” in the condition being treated if there is at least 10% improvement, preferably at least 25%, more preferably at least 50%, even more preferably at least 75%, and most preferably at least 100%. The change can be based on improvements in the severity of the treated condition in an individual, or on a difference in the frequency of improved conditions in populations of individuals with and without treatment with the compositions of the present invention.

An “effective amount” is an amount of a composition or substance(s) sufficient to effect beneficial or desired results in the treatment of hand or wrist discomfort after one or more administrations of that amount. An effective amount can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

“Hand or wrist discomfort,” as used herein, refers to symptoms involving the hand or wrist that prevent the normal functioning or use of the hand or wrist. These symptoms can be those presently indicated as manifestations or any condition that an individual finds to be non-conducive to the optimal or normal use of the hand or wrist. Such symptoms include, but are not limited to, pain, soreness, stiffness and/or general discomfort of the hand or wrist area. They include symptoms due to abnormalities in muscles, ligaments, nerves, bones and/or joints in the hand or wrist area. Symptoms can also be the result of degenerative or inflammatory diseases, tissue wear and tear, injury, tumors, infections and/or congenital abnormalities that are known, believed, or thought to affect the normal or comfortable functioning of the hand or wrist.

“Individual,” as used herein, refers to a vertebrate, preferably a mammal, more preferably a human.

A “botanical group,” as used herein, refers to a group of botanical entities that are capable of providing similar physiological effect(s) in the compositions of the invention. These botanical entities may or may not belong to the same botanical classification (such as genus, family).

“Baishaoyao,” as used herein, refers to extracts of baishaoyao. It is also known as radix paeoniae lactiflorae and paeonia lactiflora Pall. A member of the botanical group Paeonia is a substance that is capable of providing a similar physiological effect(s) as that provided by baishaoyao in the compositions of the invention, and is preferably selected from a group comprising Radix paeoniae lactiflorae; Radix paeoniae rubra; Radix paeoniae veitchii; Paeonia lactiflora Pall; Paeonia albriflora Pall; Paeonia veitchii Lynch; Paeonia lactiflora Pall. Var. trichocarpa (Bunge) Stem.; Paeonia obovata. Maxim; Paeonia anomala; Paeonia hybrida Pall; Paeonia veitchii var woodwardii; Paeonia edulis (Salisb.); Paeonia officinalis Thunb.

“Chuanxiong,” as used herein, refers to extracts of chuanxiong. It is also known as radix ligustici chuanxiong and ligusticum chuanxiong Hort. A member of the botanical group Ligusticum is a substance that is capable of providing a similar physiological effect(s) as that provided by chuanxiong in the compositions of the invention, and is preferably selected from a group comprising Radix ligustici chuanxiog; Ligusticum chuanxiong Hort; Ligusticum wallichii Franch; Cnidium officinale Makino; Ligusticum chuanxiong (S. H. Qiu. et al); Ligusticum sinense; Ligusticum chinensis; Ligustrum indicum; Ligustrum microcarpum; Ligustrum sinense; Ligustrum sinense var. stauntonii; Ligustrum stauntonii; Ligustrum villosum; Phillyrea indica.

“Fangfeng,” as used herein, refers to extracts of fangfeng. It is also known as radix ledebouriella divaricatae and ledebouriella divaricata (Turcz.). A member of the botanical group Ledebouriella is a substance that is capable of providing a similar physiological effect(s) as that provided by fangfeng in the compositions of the invention, and is preferably selected from a group comprising Radix ledebouriella divaricatae; Ledebouriella divaricate; Ledebouriella seseloides; Saposhnikovia divaricata (Turez)Schischk.; Ligusticum brachy lobum Franch; Ledebouriella divaricate (Benth. Et. Hook f.) Hiroe; Seseli delavayi Franch; Siler divaricatum (Turcz).Benth. et. Hook. f.; Seseli mairei Wolff; Seseli yunnanense Franch; Carum carvi L.; Stenocoelium divaricatum.

“Guizi,” as used herein, refers to extracts of guizi. It is also known as ramulus cinnamomi cassiae and cinnamomum cassia blume. A member of the botanical group Cinnamomum is a substance that is capable of providing a similar physiological effect(s) as that provided by guizi in the compositions of the invention, and is preferably selected from a group comprising Ramulus cinnamomi cassiae; Cinnamomum cassia Blume; Cinnamomum cassia Presl.; Cinnamomum loureiri Ness.; Cinnamomum zeylanicum Blume; Cinnamomum argenteum Gamble.; Cinnamomum tamala Nees. et. Eberm; Cinnamomum wilsonii Gamble; Cinnamomum japonicum Sieb; Cinnamomum pedunculatum Ness; Cinnamomum burmanni (Ness)Blum; Cinnamomum mairei Levl; Cinnamomum chingii Metealf; Cinnamomum chekiangense Nakai.; Cinnamomum camphora; Laurus camphora (L.); Camphora officinarum.; Laurus camphora.; Laurus camphora; Camphora camphora.

“Honghua,” as used herein, refers to extracts of honghua. It is also known as flos carthami tinctorii and carthamus tinctorius L. A member of the botanical group Carthamus is a substance that is capable of providing a similar physiological effect(s) as that provided by honghua in the compositions of the invention, and is preferably selected from a group comprising Flos carthami tinctorii; Carthamus tinctorius L.; Carthamus tinctorius; Carthamnus tinctorius; Crocus sativus L.

“Yanhusuo,” as used herein, refers to extracts of the herb yanhusuo. It is also known as rhizoma corydalis yanhusuo and corydalis yanhusuo W. T. Wang. A member of the botanical group Corydalis is a substance that is capable of providing a similar physiological effect(s) as that provided by yanhusuo in the compositions of the invention, and is preferably selected from a group comprising Rhizoma corydalis yanhusuo; Corydalis yanhusuo W. T. Wang.; Corydalis yanhusuo W. T. Wang (yanhusuo); Corydalis amabilis Migo; Corydalis amabigua Cham. et Schlecht. Var amurensts Maxim; Corydalis turtschaninovii Bess.; Corydalis turtschaninovii f. yanhusuo; Corydalis schangini (Pall.) B.; Corydalis glaucescens Rgl.; Corydalis ledebouriana Kar. Et Kir.

“Dangquiwei,”as used herein, refers to extracts of dangquiwei. It is also known as extremitas radix angelicae sinensis and angelica sinensis (liv) Diels. A member of the botanical group Angelica is a substance that is capable of providing a similar physiological effect(s) as that provided by dangquiwei in the compositions of the invention, and is preferably selected from a group comprising Radix angelica sinensis; Angelica sinensis (Oliv.) Diels.; Angelica acutiloba (s.et.Z.) Kitag; Angelica polymorpha Maxim. Var. sinensis Oliv.; Angelica polymorpha sinensis.; Angelica polymorpha sinensis; Angelica polymorpha sinensis (Oliv.); Angelica. polymorpha var. sinensis; Levisticum officinale Koch; Ligusticum acutilobum S.et Z.

“Gancao,” as used herein, refers to extracts of gancao. It is also known as radix glycyrrhizae uralensis and glycyrrhiza uralensis Fischer. A member of the botanical group Glycyrrhiza is a substance that is capable of providing a similar physiological effect(s) as that provided by gancao in the compositions of the invention, and is preferably selected from a group comprising Radix glycyrrhizae uralensis; Glycyrrhiza uralensis Fischer; Glycyrrhiza uralensis Fisch (Gan cao); Glycyrrhiza glabra L.; Glycyrrhizainflata Batal.; Glycyrrihiza korshiskyi G. Hrig.; Glycyrrhiza aspera Pall; Glycyrrhiza yunnanensis Cheng f. et L. K. Tai.; Glycyrrhiza malensis.

“Ginger,” as used herein, refers to extracts of ginger. It is also known as rhizoma zingiberis officinalis recens and zingiber officinale Rosc. A member of the botanical group Gingiberis is a substance that is capable of providing a similar physiological effect(s) as that provided by ginger in the compositions of the invention, and is preferably selected from a group comprising Rhizoma zingiberis officinalis recens.; Zingiber officinale Rosc.

“Huangqi,” as used herein, refers to extracts of hungqi. It is also known as radix astragali membranaceus and astragalus membranaceus (Fisch) Bge. A member of the botanical group Astragalus is a substance that is capable of providing a similar physiological effect(s) as that provided by huangqi in the compositions of the invention, and is preferably selected from a group comprising Radix astragali membranaceus; Astragalus membranaceus (Fisch)Bge; Astragalus membranaceus; Astragalus mongholicus Bunge; Astragalus tongolensis Ulbr.; Astragalus chrysopterus Bunge; Astragalus floridus Benth; Astragalus maonwenensis Hsiao; Astragalus yunnanensis Franch; Astragalus ernestii Comb.; Hedysarum polybotrys Hand-Mazz; Hedysarum austrosibiricum B.; Hedysarum limprichtii Ulbr.; Hedysarum viciodes Turez var taipeicum (Hand-Mazz) Lin.; Hedysarum sikkimense Benth.

“Sangzhi,” as used herein, refers to extracts of sangzhi. It is also known as Ramulus mori albac and mori albae. A member of the botanical group Morus is a substance that is capable of providing a similar physiological effect(s) as that provided by sangzhi in the compositions of the invention, and is preferably selected from a group comprising Folium mori albae; Morus alba L.; Morus australis; Morus australis Poir; Morus mongolica Schneid; Morus cathayana Hemsl.; Morus laevigata wall; Morus acidosa (Griff.); Morus alba stylosa ((Ser.)Bur.); Morus indica (non L.); Morus stylosa (Ser.); Morus alba; Morus alba f. tatarica; Mortus alba var. constantinopolitana; Morus alba var. multicaulis; Morus alba var. stylosa; Morus alba var. tatarica; Morus constantinopolitana; Morus multicaulis; Morus tatarica.

“Yinyanghuo,” as used herein, refers to extracts of yinyanghuo. It is also known as herba epimedii and epimedium grondiflorum Morr. A member of the botanical group Epimedium is a substance that is capable of providing a similar physiological effect(s) as that provided by yinyanghuo in the compositions of the invention, and is preferably selected from a group comprising Herba epimedii; Epimedium grondiflorum Morr.; Epimedium macranthum Morr et. Decne; Epimedium sagittatum(S.et.Z.)Maxim; Epimedium brevicomum Maxim; Epimediumacuminatum Franch; Epimedium elongatum Komar; Epimedium koreanum Nakai; Epimedium pubescens Maxim; Epimedium macranthum; Epimedium violaceum; Aceranthus sagittatum.

“Weilingxian,” as used herein, refers to extracts of weilingxian. It is also known as radix clematidis and clematis chinensis osbeck C. A member of the botanical group Clematis is a substance that is capable of providing a similar physiological effect(s) as that provided by weilingxian in the compositions of the invention, and is preferably selected from a group comprising Radix clematidis chinensis; Radix clematidis uncinatae; Radix clematidis manshurica; Radix clematidis hexapetalae; Clematis chinensis osbeck C.; Clematis hexapetale Pall; Clematis uncinata Champ; Clematis manshurica Rupr; Clematis angustifolia Jacq.; Clematis finetiana Levl. Et. Vant; Clematis povoliniana Pamp. Clematis.

“Dazao,” as used herein, refers to extracts of dazao. It is also known as fructus zizypho jujubae and ziziphus jujuba Mill. A member of the botanical group Zizyphus is a substance that is capable of providing a similar physiological effect(s) as that provided by dazao in the compositions of the invention, and is preferably selected from a group comprising Fructus zizypho jujubae; Zizyphus jujuba Mill; Zizyphus jujuba Mill var inermis (Bunge) Rehd; Ziziphus jujuba; Ziziphus sativa (Gaertn.); Ziziphus vulgaris (Lam.); Ziziphus zizyphus ((L.)Karsten.); Ziziphus mauritiana; Ziziphus spinosa; Ziziphus vulgaris var. spinosa; Zizyphus jujuba; Zizyphus lotos; Zizyphus sativa; Zizyphus vulgaris; Zizyphus zizyphus; Rhamnus jujuba; Rhamnus zizyphus.

“Vitamin B-1,” as used herein, refers to the thiamine member of the Vitamin B series or complex.

“Vitamin B-2,” as used herein, refers to the riboflavin member of the Vitamin B series or complex.

“Vitamin B-3,” as used herein, refers to the niacin member of the Vitamin B series or complex.

“Vitamin B-5,” as used herein, refers to the pantothenic acid member of the Vitamin B series or complex.

“Vitamin B-6,” as used herein, refers to the pyridoxine member of the Vitamin B series or complex.

“Vitamin C,” as used herein, refers to ascorbic acid and salts thereof.

“Vitamin E,” as used herein, refers to D alpha-tocopherol, preferably in its succinate form.

“Beta-carotene,” as used herein, refers to the form(s) of Vitamin A known to persons of skill in the art as such.

“Zinc,” as used herein, refers to the form(s) of the mineral known to persons of skill in the art to be therapeutically effective in the body of the individual. It is preferably provided as zinc gluconate.

“Quercetin,” as used herein, refers to the substance known in the art to result from hydrolysis of quercitron. Quercitron can be obtained from sources known in the art, such as quercitron bark.

“Selenium,” as used herein, refers to the form(s) of the mineral known to persons of skill in the art to be therapeutically effective in the body of the individual. It is preferably provided as selenium aminoate.

“Rice flour powder,” as used herein, refers to the additive known to persons of skill in the art as such.

“Magnesium,” as used herein, refers to the form(s) of the mineral known to persons of skill in the art to be therapeutically effective in the body of the individual. It is preferably provided as magnesium stearate NT.

“Extract,” as used herein, refers to the substances obtained from the specified source plant, or parts thereof (for e.g., root, bark, leaves). Any method of extraction that yields extracts that retain the biological activity of the substances contained in the extract source can be used to produce extracts used in this invention. Preferably, the ingredients of the compositions of the present invention are extracted as an aqueous solution. The extraction is preferably performed under conditions of high pressure, preferably from 0.5 to 12 bar, more preferably 1 to 10 bar, most preferably 3 to 7 bar, and preferably at elevated temperatures (preferably within a range of 15° C. to 120° C., more preferably 30° C. to 100° C., most preferably 45° C. to 75° C.). The extract is preferably treated to yield a form suitable for mixing of two or more substances. The form is preferably a dried powder. The powder form is yielded from preferably at least about a 1:10, more preferably at least about a 1:8, most preferably at least about a 1:5 concentrate of the starting solution. Concentration to powder form is preferably achieved by evaporation to yield a dried powder form. The extracts used in this invention can also be obtained from commercial sources such as Sun Ten Laboratories (Irvine, Calif.), Qualiherb (Cerritos, Calif.), Mayway (Oakland, Calif.), Ming Tong Herb (Oakland, Calif.) and Acta (Sunnyvale, Calif.). It is understood that any method or conditions known in the art to yield extracts comparable in therapeutic effectiveness to those produced by the preceding preferred extraction method can be used for the purposes of this invention.

### Formulation of the Compositions

Each substance contained in the compositions provided by this invention is provided in an amount that lies within specific quantitative ranges herein disclosed to be effective for treating hand or wrist discomfort.

According to the present invention, an effective amount of a composition comprises preferably from 20 mg to 400 mg, more preferably 80 mg to 300 mg, most preferably 120 mg to 200 mg of a member of the botanical group Paeonia (such as baishaoyao); preferably 20 mg to 400 mg, more preferably 80 mg to 300 mg, most preferably 120 mg to 200 mg of a member of the botanical group Ledebouriella (such as fangfeng); preferably 40 mg to 600 mg, more preferably 120 mg to 500 mg, most preferably 250 mg to 400 mg of a member of the botanical group Cinnamomum (such as guizi); preferably 20 mg to 400 mg, more preferably 80 mg to 300 mg, most preferably 120 mg to 200 mg of a member of the botanical group Carthamus (such as honghua); preferably 40 mg to 600 mg, more preferably 120 mg to 500 mg, most preferably 250 mg to 400 mg of a member of the botanical group Ligusticum (such as chuanxiong); and/or preferably 40 mg to 600 mg, more preferably 120 mg to 500 mg, most preferably 250 mg to 400 mg of a member of the botanical group Corydalis (such as yuanhusuo).

In the various embodiments of the invention, the compositions further comprise preferably from 40 mg to 600 mg, more preferably 120 mg to 500 mg, most preferably 250 mg to 400 mg of a member of the botanical group Angelica (such as dangquiwei); preferably 20 mg to 400 mg, more preferably 80 mg to 300 mg, most preferably 120 mg to 200 mg of a member of the botanical group Glycyrrhiza (such as gancao); preferably 20 mg to 400 mg, more preferably 80 mg to 300 mg, most preferably 120 mg to 200 mg of a member of the botanical group Gingiberis (such as ginger); preferably 100 mg to 1500 mg, more preferably 300 mg to 1200 mg, most preferably 600 mg to 900 mg of a member of the botanical group Astragalus (such as huangqi); preferably 50 mg to 800 mg, more preferably 150 mg to 650 mg, most preferably 300 mg to 500 mg of a member of the botanical group Morus (such as sangzhi); preferably 20 mg to 400 mg, more preferably 80 mg to 300 mg, most preferably 120 mg to 200 mg of a member of the botanical group Epimedium (such as yinyanghuo); preferably 50 mg to 800 mg, more preferably 150 mg to 650 mg, most preferably 300 mg to 500 mg of a member of the botanical group Clematis (such as weilingxian); preferably 10 mg to 200 mg, more preferably 20 mg to 150 mg, most preferably 50 mg to 100 mg of a member of the botanical group Zizyphus (such as dazao); preferably 3 mg to 50 mg, more preferably 8 mg to 40 mg, most preferably 15 mg to 30 mg vitamin B-1; preferably 3 mg to 50 mg, more preferably 8 mg to 40 mg, most preferably 15 mg to 30 mg vitamin B-2; preferably 5 mg to 100 mg, more preferably 25 mg to 75 mg, most preferably 50 mg to 60 mg vitamin B-3; preferably 3 mg to 50 mg, more preferably 8 mg to 40 mg, most preferably 15 mg to 30 mg vitamin B-S; preferably 20 mg to 400 mg, more preferably 80 mg to 300 mg, most preferably 120 mg to 200 mg vitamin B-6; preferably 80 mg to 1200 mg, more preferably 200 mg to 1000 mg, most preferably 350 mg to 750 mg vitamin C; preferably 10 mg to 160 mg, more preferably 35 mg to 120 mg, most preferably 60 mg to 90 mg vitamin E; preferably 6 mg to 85 mg, more preferably 10 mg to 60 mg, most preferably 30 mg to 50 mg beta-carotene; preferably 15 mg to 250 mg, more preferably 50 mg to 200 mg, most preferably 100 mg to 150 mg zinc; preferably 5 mg to 100 mg, more preferably 25 mg to 75 mg, most preferably 50 mg to 60 mg quercetin; preferably 5 mg to 80 mg, more preferably 15 mg to 60 mg, most preferably 40 mg to 50 mg selenium; preferably 8 mg to 120 mg, more preferably 25 mg to 100 mg, most preferably 50 mg to 75 mg rice flour powder; and/or preferably 8 mg to 120 mg, more preferably 25 mg to 100 mg, most preferably 50 mg to 75 mg magnesium.

Selection of suitable members of a particular botanical group to be included in a composition can be achieved using methods known in the art. For example, a suitable member of the botanical group Paeonia would be expected to be capable of providing a similar physiological effect(s) as that provided by baishaoyao in a composition of the invention. Such a member can be selected based on, for example, whether it is known, shown and/or suspected to possess said similar physiological effect(s). Thus, for example, a determination of whether a candidate substance can be a member of the botanical group Paeonia can be done based on, for example, a similar pharmacological or medicinal classification for both the candidate substance and baishaoyao. However, the activity and/or function provided by a particular substance, such as baishaoyao, need not be identified or specified. A determination of whether a candidate substance can be a member of a particular botanical group, for example the Paeonia group, can also be empirical, for example, by substituting said candidate substance for baishaoyao in a composition, and assessing the relevant therapeutic effect(s) of the composition. Such a determination can be done using methods and techniques known in the art.

According to this invention, the compositions can be formulated in whatever form that retains the efficacy of the composition for treating hand or wrist discomfort. Preferably, the compositions are packaged in the form of capsules. The capsules are preferably of size “0”, “00”, “000”, “1”, “2”, “3” or “4.” A preferred method for packaging into capsules involves mixing substances (extracts, vitamin and minerals) that are preferably in powder form. The substances are preferably mixed to at least 30%, more preferably to at least 60%, even more preferably to at least 90% mixture consistency, and most preferably to homogeneity. The substances in powder form are provided in the initial mixture at ratios according to the effective quantities disclosed above. Methods for mixing the substances are known in the art, including, but not limited to, stirring, agitation or vibration achieved manually or through the aid of a machine. A preferred mixing machine is a V-mixer, preferably of 100 to 1400-liter size, more preferably of 150 to 1300-liter size, and most preferably of 200 to 1200-liter size. Preferably, the resulting powder mixture is filtered to screen out particulates (i.e., anything that a person of skill in the art would recognize to be larger than powder size). A preferred filter is a 1/20-inch particle size filter. Preferably, the filtered mixture is packaged into capsules according to the weight desired for each capsule. Preferably, the capsule is of size “00”. The weight of mixture per capsule is preferably from 5 mg to 1000 mg, more preferably 100 mg to 800 mg, even more preferably 400 mg to 700 mg. It is understood that other physical forms of the compositions of this invention suitable for administration to an individual can also be used, including, for example, tablets, salves or liquids, as long as the compositions can be delivered to the target tissues in the body where the compositions in the preferred form described above exert their effects.

The ingredients of the compositions can be mixed with pharmaceutically acceptable solvents, excipients and/or filler substances. These materials are known in the art, and are described in sources such as Remington's Pharmaceutical Sciences, 18th Edition, Mack Publishing (1990).

### Administration of Compositions

Compositions in any of the physical forms described above can be administered by any method known to one of skill in the art, but oral administration is preferred. The compositions are preferably administered in capsule form.

An effective amount of a composition is provided preferably in from 1 to 8 administrations, more preferably in from 2 to 6 administrations, and most preferably in from 3 to 5 administrations. Administration of an effective amount is preferably completed within 24 hours. A composition can be ingested alone, or with any other substance, such as a liquid, that aids ingestion of the compositions. Ingestion of the compositions can be before or after food consumption.

### EXAMPLES

### Example 1

### An Illustrative Example of the Formulation of A Single “00” Capsule, and the Production Thereof

A composition shown to be effective for treating hand or wrist discomfort contained substances in the indicated quantities as listed in Table 1.

TABLE 1

| SUBSTANCE [commercial source] | AMOUNT (mg) |
|---|---|
| Baishaoyao [Qualiherb, Cerritos, CA] | 33.3 |
| Fangfeng [Qualiherb, Cerritos, CA] | 33.3 |
| Guizi [Qualiherb, Cerritos, CA] | 50 |
| Honghua [Qualiherb, Cerritos, CA] | 33.3 |
| Chuanxiong [Qualiherb, Cerritos, CA] | 50 |
| Yuanhusuo [Mayway, Oakland, CA] | 50 |
| Dangquiwei [Mayway, Oakland, CA] | 50 |
| Gancao [Qualiherb, Cerritos, CA] | 33.3 |
| Ginger [Qualiherb, Cerritos, CA] | 33.3 |
| Huangqi [Mayway, Oakland, CA] | 133 |
| Sangzhi [Qualiherb, Cerritos, CA] | 66.7 |
| Yinyanghuo [Qualiherb, Cerritos, CA] | 33.3 |
| Weilingxian [Mayway, Oakland, CA] | 66.7 |
| Dazao [Mayway, Oakland, CA] | 16.7 |
| Vitamin B-1 (Thiamine) [Acta, | 4.2 |
| Sunnyvale, CA] | |
| Vitamin B-2 (Riboflavin) [Acta, | 4.2 |
| Sunnyvale, CA] | |
| Vitamin B-3 (Niacin) [Acta, Sunnyvale, | 8.3 |
| CA] | |
| Vitamin B-5 (Pantothenic acid) [Acta, | 4.2 |
| Sunnyvale, CA] | |
| Vitamin B-6 (pyridoxine) [Acta, | 33 |
| Sunnyvale, CA] | |
| Vitamin C (ascorbic acid) [Acta, | 100 |
| Sunnyvale, CA] | |
| Vitamin E (D alpha-tocopherol | 14.1 |
| succinate) [Acta, Sunnyvale, CA] | |
| Beta-carotene [Acta, Sunnyvale, CA] | 7.5 |
| Zinc gluconate [Acta, Sunnyvale, CA] | 21 |
| Quercetin [Acta, Sunnyvale, CA] | 8.3 |
| Selenium aminoate [Acta, Sunnyvale, | 6.7 |
| CA] | |
| Rice flour powder [Acta, Sunnyvale, | 10 |
| CA] | |
| Magnesium stearate NT [Acta, | 10 |
| Sunnyvale, CA] | |

Capsules containing the composition above were manufactured according to the method used by the commercial manufacturer, Acta (Sunnyvale, Calif.). Briefly, the substances listed above, in powder form and obtained from the commercial sources indicated, were mixed in input amounts in accordance to the ratio of the substances in the composition as a whole. Mixing was accomplished with a V-mixer, grinding for 15 to 30 minutes, at a speed of 15 to 30 rpm (rounds per minute), to produce a homogenous mixture of the input substances. Particulates (non-powder forms) were then filtered out with a 1/20-inch particle size filter that separated particulates from the powder. 698.3 mg of the filtered mixture was then packaged into each size “00” capsule.

### Example 2

### A First Illustrative Example of Treatment of Wrist Discomfort

An individual complaining of wrist pain was asked to rate the severity of her symptom on a scale of 1 to 5, 1 being normal condition and 5 being severe pain. The individual was instructed to assess frequency of pain (e.g., occasional, intermittent, or constant) as a primary criterion for pain severity. The composition of Example 1 was then administered at 2 capsules per dosage for the first 5 dosages, followed by 1 capsule per dosage for another 7 dosages, 3 dosages daily.

TABLE 2
Severity of Wrist Pain

| Before | Dose | Dose | Dose |
|---|---|---|---|
| Use | 1-10 | 11 | 12 |
| 4 | 4 | 3 | 3 |

As shown in Table 2, starting at a severity of pain of 4 on the 1 to 5 scale, administration of the composition of Example 1 resulted in a clear decrease to a severity of 3 after 10 dosages (about 3 days). This result shows that the composition of Example 1 provides a therapeutic effect in the treatment of wrist pain.

The therapeutic effects of the compositions of this invention can also be demonstrated in the treatment of hand discomfort in accordance with the foregoing or a similar study protocol.

### Example 3

### A Second Illustrative Example of Treatment of Hand Or Wrist Discomfort

A randomized, double-blinded and controlled study is performed. Control groups use either an existing treatment (such as non-steroidal anti-inflammatory drugs or aspirin) or a sugar pill placebo. Dosage amount and schedule are 1 to 6 capsules (of the composition of Example 1) daily. At least 20 individuals are tested, randomly assigned in approximately equal number to treatment (administered the composition of Example 1) and control groups. Individuals are evaluated as suffering from a particular hand or wrist discomfort symptom, such as pain and/or soreness of the hand and/or wrist.

The study is carried out for at least 1 week. During the treatment course, a dosage amount selected from the range of 1 to 6 capsules is administered to each individual once or multiple times daily, not exceeding 6 capsules per day. Capsules are administered before or after food consumption.

A clinical questionnaire is used to evaluate individuals' hand or wrist symptoms. Frequency of pain/soreness (e.g., occasional, intermittent, or constant) is used as a primary criterion for assessing pain/soreness severity. A clinical coordinator and/or physician evaluates the individual's hand or wrist discomfort symptoms and fills out the questionnaire. Evaluations can be performed daily, or more or less frequently depending on statistical or clinical (ability to detect or track symptomatic improvements) needs.

Assessment of symptoms is divided into 4 grades: (1) clinical cure, as defined as free of symptoms; (2) significant efficacy, as defined as significantly improved symptoms (e.g., reduction of more than 3 points on any one of discomfort scoring scales); (3) efficacy, as defined as partially improved (e.g. reduction of more than 1 point on any one of discomfort scoring scales) symptoms; and (4) non-efficacy, as defined as no improvement in symptoms.

### Example 4

### A Third Illustrative Example of Treatment of Hand or Wrist Discomfort

10 individuals complaining of wrist pain or elbow pain (which can be part of hand pain) were asked to rate the severity of their symptom on a scale of 1 to 10, 1 being normal condition and 10 being severe pain. The individuals were instructed to assess frequency of pain (e.g., occasional, intermittent, or constant) as a primary criterion for pain severity. The composition of Example 1 was then administered in conjunction with an enhancing composition (which by itself is not efficacious in treating hand or wrist discomfort conditions), which is disclosed in co-pending application entitled “Compositions And Methods For Enhancing Therapeutic Effects” (U.S. Provisional Application Ser. No. 60/208,990; U.S. patent application Ser. No. ______, filed even date herewith), which is hereby incorporated in its entirety by reference. Each capsule containing the enhancing composition contained substances described in Table 3 below.

TABLE 3

| SUBSTANCE [Commercial Source] | AMOUNT (mg) |
|---|---|
| Xuejie [Min Tong Herb, Oakland, CA] | 133 |
| Yanhusuo [Mayway, Oakland, CA] | 83 |
| Baishaoyao [Qualiherb, Cerritos, CA] | 83 |
| Shanqi [Sun Ten, Irvine, CA] | 250 |
| Gancao [Qualiherb, Cerritos, CA] | 83 |
| White willow bark [Acta, Sunnyvale, | 10 |
| CA] | |
| Black cohosh root [Acta, Sunnyvale, | 10 |
| CA] | |
| L-carnitine [Acta, Sunnyvale, CA] | 50 |
| Vitamin E (D Alpha-Tocopherol | 21.19 |
| succinate) [Acta, Sunnyvale, CA] | |
| Vitamin C [Acta, Sunnyvale, CA] | 30 |

Individuals were administered various numbers of capsules (as indicated in the data table below) of the composition of Example 1 and the enhancing composition of Table 3. The individual was asked to assess her symptom daily using the 0 to 10 scale for the duration of the study. A therapeutic effect is obtained if there is a decrease in the severity of the symptom.

The results for treatment of wrist pain are described in Table 4 below.

TABLE 4

| | Before | | | | |
|---|---|---|---|---|---|
| | treatment | 1-3 days | 4-6 days | 7-10 days | 2 weeks |
| Wrist pain | 8.8 | 5.8 | 4 | 2.2 | 0 |
| (symptom | | | | | |
| score) | | | | | |
| H&W (average | | 6 | 3.7 | 3 | 2 |
| capsules/day) | | | | | |
| Enh. (average | | 3.5 | 2.5 | 2 | 1.5 |
| capsules/day) | | | | | |

H&W: Composition of Example 1

Enh.: Composition of Table 3

The results for treatment of elbow pain are described in Table 5 below.

TABLE 5

| | Before | | | | |
|---|---|---|---|---|---|
| | treatment | 1-3 days | 4-6 days | 7-10 days | 2 weeks |
| Elbow pain | 9 | 6 | 3 | 0 | 0 |
| (symptom | | | | | |
| score) | | | | | |
| H&W (average | | 6 | 3 | 3 | 0 |
| capsules/day) | | | | | |
| Enh. (average | | 4 | 0 | 2 | 0 |
| capsules/day) | | | | | |

H&W Composition of Example 1

Enh.: Composition of Table 3

The results indicate a significant therapeutic effect with respect to wrist and elbow pain in individuals who were administered the composition of Example 1.

Although the foregoing invention has been described in some detail by way of illustration and example for purposes of clarity of understanding, it will be apparent to those skilled in the art that certain changes and modifications can be practiced. Therefore, the description and examples should not be construed as limiting the scope of the invention, which is delineated by the appended claims.

1. A composition for treating hand or wrist discomfort comprising 2 substances selected from the group consisting of a member of the botanical group Paeonia, a member of the botanical group Ligusticum, a member of the botanical group Ledebouriella, a member of the botanical group Cinnamomum, a member of the botanical group Carthamus and a member of the botanical group Corydalis.

2. A composition for treating hand or wrist discomfort comprising 3 substances selected from the group consisting of a member of the botanical group Paeonia, a member of the botanical group Ligusticum, a member of the botanical group Ledebouriella, a member of the botanical group Cinnamomum, a member of the botanical group Carthamus and a member of the botanical group Corydalis.

3. A composition for treating hand or wrist discomfort comprising 4 substances selected from the group consisting of a member of the botanical group Paeonia, a member of the botanical group Ligusticum, a member of the botanical group Ledebouriella, a member of the botanical group Cinnamomum, a member of the botanical group Carthamus and a member of the botanical group Corydalis.

4. A composition for treating hand or wrist discomfort comprising 5 substances selected from the group consisting of a member of the botanical group Paeonia, a member of the botanical group Ligusticum, a member of the botanical group Ledebouriella, a member of the botanical group Cinnamomum, a member of the botanical group Carthamus and a member of the botanical group Corydalis.

5. A composition for treating hand or wrist discomfort comprising a member of the botanical group Paeonia, a member of the botanical group Ligusticum, a member of the botanical group Ledebouriella, a member of the botanical group Cinnamomum, a member of the botanical group Carthamus and a member of the botanical group Corydalis.

6. A composition selected from the group consisting of the compositions of claims 1-5, further comprising a substance selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

7. A composition selected from the group consisting of the compositions of claims 1-5, further comprising 2 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

8. A composition selected from the group consisting of the compositions of claims 1-5, further comprising 3 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

9. A composition selected from the group consisting of the compositions of claims 1-5, further comprising 4 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

10. A composition selected from the group consisting of the compositions of claims 1-5, further comprising 5 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

11. A composition selected from the group consisting of the compositions of claims 1-5, further comprising 6 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

12. A composition selected from the group consisting of the compositions of claims 1-5, further comprising 7 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

13. A composition selected from the group consisting of the compositions of claims 1-5, further comprising 8 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

14. A composition selected from the group consisting of the compositions of claims 1-5, further comprising 9 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-i, vitamin B-2, vitamin B-3, vitamin B-5, vitamin B-6, vitamin C, vitamin E, beta-carotene, zinc, quercetin, selenium, rice flour powder and magnesium.

15. A composition selected from the group consisting of the compositions of claims 1-5, further comprising 10 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

16. A composition selected from the group consisting of the compositions of claims 1-5, further comprising 11 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

17. A composition selected from the group consisting of the compositions of claims 1-5, further comprising 12 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

18. A composition selected from the group consisting of the compositions of claims 1-5, further comprising 13 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

19. A composition selected from the group consisting of the compositions of claims 1-5, further comprising 14 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

20. A composition selected from the group consisting of the compositions of claims 1-5, further comprising 15 substances selected from the group consisting of dangquiwei, gancao, ginger, huangqi, sangzhi, yinyanghuo, weilingxian, dazao, vitamin B-1, vitamin B-2, vitamin B-3, vitamin B-5, vitamin B-6, vitamin C, vitamin E, beta-carotene, zinc, quercetin, selenium, rice flour powder and magnesium.

21. A composition selected from the group consisting of the compositions of claims 1-5, further comprising 16 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quereetin, selenium, rice flour powder and magnesium.

22. A composition selected from the group consisting of the compositions of claims 1-5, further comprising 17 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

23. A composition selected from the group consisting of the compositions of claims 1-5, further comprising 18 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

24. A composition selected from the group consisting of the compositions of claims 1-5, further comprising 19 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

25. A composition selected from the group consisting of the compositions of claims 1-5, further comprising 20 substances selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

26. A composition selected from the group consisting of the compositions of claims 1-5, further comprising a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.

27. The composition of any of claims 1-5, wherein the member of the botanical group Paeonia is baishaoyao, the member of the botanical group Ligusticum is chuanxiong, the member of the botanical group Ledebouriella is fangfeng, the member of the botanical group Cinnamomum is guizi, the member of the botanical group Carthamus is honghua and the member of the botanical group Corydalis is yanhusuo.

28. The composition of claim 6, wherein the member of the botanical group Paeonia is baishaoyao, the member of the botanical group Ligusticum is chuanxiong, the member of the botanical group Ledebouriella is fangfeng, the member of the botanical group Cinnamomum is guizi, the member of the botanical group Carthamus is honghua, the member of the botanical group Corydalis is yanhusuo, the member of the botanical group Angelica is dangquiwei, the member of the botanical group Glycyrrhiza is gancao, the member of the botanical group Gingiberis is ginger, the member of the botanical group Astragalus is huangqi, the member of the botanical group Morus is sangzhi, the member of the botanical group Epimedium is yinyangho, the member of the botanical group Clematis is weilingxian, and the member of the botanical group Zizyphus is dazao.

29. A method of treating hand or wrist discomfort comprising administering to the individual an effective amount of a composition selected from the group consisting of the compositions of claims 1 to 5.

30. A method of making a composition for treating hand or wrist discomfort comprising combining at least two substances selected from the group consisting of a member of the botanical group Paeonia, a member of the botanical group Ligusticum, a member of the botanical group Ledebouriella, a member of the botanical group Cinnamomum, a member of the botanical group Carthamus and a member of the botanical group Corydalis.

31. The method of claim 30, wherein said at least two substances are combined with at least one substance selected from the group consisting of a member of the botanical group Angelica, a member of the botanical group Glycyrrhiza, a member of the botanical group Gingiberis, a member of the botanical group Astragalus, a member of the botanical group Morus, a member of the botanical group Epimedium, a member of the botanical group Clematis, a member of the botanical group Zizyphus, vitamin B-1, vitamin B-2, vitamin B-3, vitamin B-5, vitamin B-6, vitamin C, vitamin E, beta-carotene, zinc, quercetin, selenium, rice flour powder and magnesium.
